(19) United States
(12) Patent Application Publication
(10) Pub. No.: US 2018/0315491 A1
(43) Pub. Date: Nov. 1, 2018

(54) SYSTEM AND METHOD FOR CUSTOMIZING AN APPLICATION USER INTERFACE OF AN OPTICAL LAB MANAGEMENT SYSTEM

(71) Applicant: ESSILOR INTERNATIONAL (COMPAGNIE GENERALE D'OPTIQUE), Charenton-le-Pont (FR)

(72) Inventors: Michael A. FILIPOVICH, Dallas, TX (US); Dan MALENIUS, Dallas, TX (US); John HIEMENZ, Dallas, TX (US); Mike ENGELS, Dallas, TX (US); Mark GOLDMAN, Dallas, TX (US); Adam SWANSON, Dallas, TX (US); Jill DELONG, Dallas, TX (US)

(73) Assignee: Essilor International, Charenton-Le-Pont (FR)

(21) Appl. No.: 15/768,222

(22) PCT Filed: Oct. 16, 2015
(86) PCT No.: PCT/EP2015/074056

§ 371 Date: Apr. 13, 2018

(51) Int. Cl. G16H 10/40; G06F 17/30; G16H 40/63

(52) U.S. Cl. CPC G16H 10/40; G06F 3/04847; G16H 40/63; G06F 17/30002

(57) ABSTRACT

Systems and methods that enable an end user to create and define an application user interface of an optical lab management system. For example, an optical lab management system includes memory configured to store computer executable instructions and data; a network interface configured to enable the optical lab management system to communicate data with at least one networked device; and a processor for executing the computer executable instructions. The computer executable instructions can include instructions for providing at least one database editor configured to enable an end user to design an application user interface of the optical lab management system. The at least one database editor includes at least one configuration field for defining a layout of the application user interface and for defining specific data fields to be displayed on the application user interface.

### BACKGROUND

The present disclosure relates generally to systems, computer-implemented methods, and computer-readable storage media that execute, are configured to execute, or store instructions that enable the customization of an application user interface of an optical lab management system. As used herein, the term application user interface refers to the look and feel as well as the function associated with a user interface.

Traditional laboratory management systems (LMSs) typically offer a pre-determined screen layout, prompt sequence and functionality that's designed by the software development team and delivered to customers following a typical life-cycle of software development. Screens may have a pleasant look, but are typically static and not amenable to change by the end users. For example, the user interface layout, prompt-sequence, and background image(s) or color are determined during software development and is generally immune to end-user personalization.

Thus, the end-users have limited ability to adapt or personalize the screen or user interface as desired. Instead, the success of the business often relies on the foresight and design abilities of the system developers. Some LMS providers are open to suggestions and change, while others are less accommodating to requests from the users of the system. However, any change request by the end user often takes time for approval, implementation, and testing. This is especially true for applications that are populated across a large network of laboratories or commercial entities. As such, labs have to adapt processes and behaviors to find ways to work around design shortcomings of an LMS.

### BRIEF SUMMARY OF THE DISCLOSED EMBODIMENTS

The disclosed embodiments include systems, computer-implemented methods, and computer-readable storage media that execute, are configured to execute, or store instructions that enable an end user of an optical lab management system to create and define an application user interface within the optical lab management system. As one example, specific embodiments disclosed herein include an optical lab management system that includes memory configured to store computer executable instructions and data; a network interface configured to enable the optical lab management system to communicate data with at least one networked device; and a processor for executing the computer executable instructions. In one embodiment, the computer executable instructions comprise instructions for providing at least one database editor configured to enable an end user to design an application user interface of the optical lab management system. The at least one database editor includes at least one configuration field for defining a layout of the application user interface and for defining specific data fields to be displayed on the application user interface.

Other aspects and advantages of the invention will be apparent from the following description and the appended claims.

### BRIEF DESCRIPTION OF THE DRAWINGS

Illustrative embodiments of the present invention are described in detail below with reference to the attached drawing figures, which are incorporated by reference herein, and wherein:

FIG. 1 illustrates a system for customizing an application user interface of an optical lab management system in accordance with one embodiment;

FIG. 2 is a block diagram conceptually illustrating components of an optical lab management system in accordance with one embodiment;

FIG. 3 illustrates an example of a Header Editor in accordance with one embodiment;

FIG. 4 illustrates an example of a Palette Editor in accordance with one embodiment;

FIG. 5 illustrates an example of a Template Field Editor in accordance with one embodiment;

FIG. 6 is a flowchart illustrating a process for customizing an application user interface of an optical lab management system in accordance with one embodiment;

FIG. 7 illustrates an example of application user interface generated in accordance with one embodiment;

FIG. 8 illustrates an example of a holiday theme applied to the application user interface shown in FIG. 7 in accordance with one embodiment;

FIG. 9 illustrates another example of an application user interface generated in accordance with one embodiment; and

FIG. 10 illustrates another example of an application user interface generated in accordance with one embodiment.

The illustrated figures are only exemplary and are not intended to assert or imply any limitation with regard to the environment, architecture, design, or process in which different embodiments may be implemented.

### DETAILED DESCRIPTION

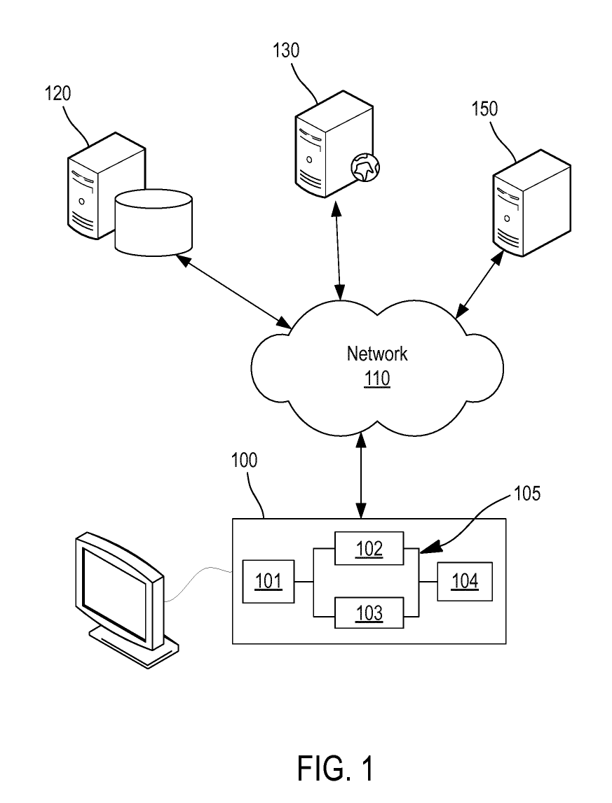

FIG. 1 illustrates a system 100 for customizing an application user interface of an optical lab management system in accordance with one embodiment. In one embodiment, the system 100 comprises components including one or more processors 101, a computer-readable storage media 102, an input/output interface 103, and a network interface 104. Each of the components of the system 100 communicates via a systems bus 105 that transfers data between the components. The processors 101 may be of any type and having any number of cores. The processors 101 are configured to process data and execute computer-executable instructions. These instructions may include, but are not limited to, machine code instructions, byte code for a software interpreter, object code, and source code in a high-level programming language.

Data and computer-executable instructions are stored in the computer-readable storage media 102. The computer-readable storage media 102 may be any appropriate memory device or computer storage media, such as, but not limited to, a hard disk drive, random access memory, read only memory, electrically erasable programmable read-only memory, flash memory or other memory technology, compact disc-read only memory, digital versatile disks or other optical disk storage, magnetic cassettes, magnetic tape, magnetic disk storage or other magnetic storage devices. In some embodiments, the data and executable instructions may be stored on a component or device that is external to the system 100 such as, but not limited to, being stored on a network device or on an external memory drive.

The input/output (I/O) interface 103 comprises an input interface for receiving user input or data from one or more peripheral devices. For example, the I/O interface 103 may receive user input or data from one or more input devices such as, but not limited to, a keyboard, mouse, touch screen, microphone, scanner, and/or a camera. The I/O interface 103 also comprises an output interface for outputting information to one or more device or component associated with the system 100. For example, the I/O interface 103 may output data or other information to a display device for displaying information to a user, another system, and/or to a printer.

The network interface 104 may include one or more wired or wireless interfaces such as, for example, an Ethernet port or a wireless transceiver for enabling the system 100 to send and receive data over a communication network 110 from one or more networked device. The network interface 104 is not limited to any particular communication protocol or hardware interface.

The communication network 110 may be any type of wired or wireless connection, which may include one or more public or private networks or some combination thereof, such as the Internet, an intranet, a mobile cellular or data network, or any other network operable to transmit data to and from the system 100.

As illustrated in FIG. 1, in certain embodiments, the system 100 may communicate over the communication network 110 with a number of network devices including, but not limited to, one or more databases 120, web servers 130, and internal or external systems 150. For example, in some embodiments, the system 100 communicates with a database 120 that stores optical lab orders. The database 120 may contain information provided and/or maintained by a single entity (e.g., by a provider of the system 100) or by a combination of entities including one or more independent third parties. In certain embodiments, the system 100 may also be configured to store all system data in the database 120. The system 100 may also be configured to communicate with a web server 130 that provides a knowledge database such as, but not limited to, VisionWeb™ or EyeFinity™. Still, in some embodiments, the system 100 may also be configured to communicate with one or more external or internal systems 150 such as, but not limited to, systems located at an optometrist office or other optical facilities, an internal employee timekeeping system, an email server, and an accounting server.

Additionally, the system 100 is not limited to any particular design platform but instead may be any type of computing device such as but not limited to a personal computer, mobile computer, laptop, smart phone, personal digital assistant, server, or any other computing device that is configured to execute the computer executable instructions of an optical lab management system in accordance with the embodiments disclosed herein.

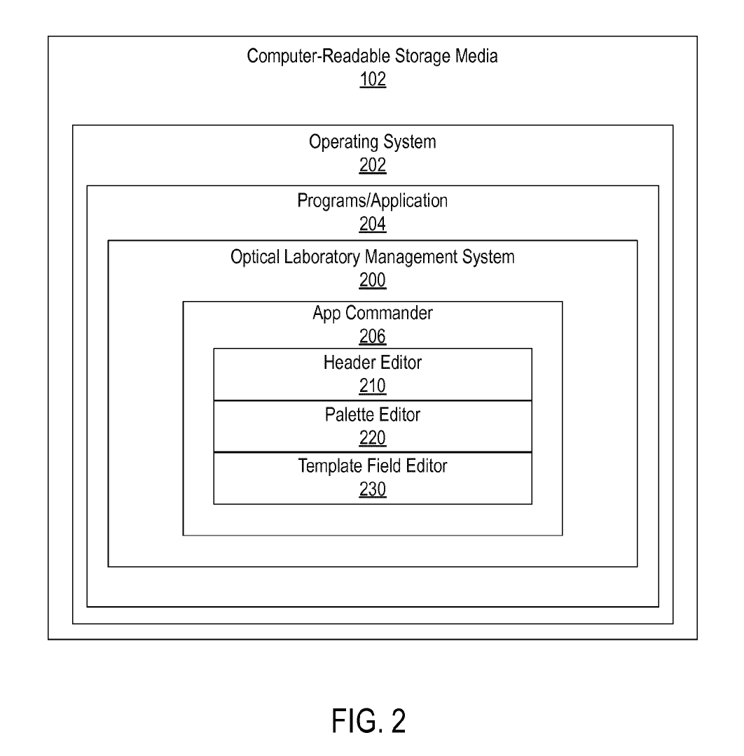

FIG. 2 is a block diagram conceptually illustrating software components of an optical lab management system in accordance with one embodiment. As previously stated, the depicted software components are stored in the computer-readable storage media 102 of the system 100. In one embodiment, the software components include an operating system 202. Non-limiting examples of operating system 202 include various versions of Linux® and Windows®. The operating system 202 provides instructions for controlling the system 100's basic functions, such as scheduling tasks, controlling peripherals, and executing programs/applications 204 such as, but not limited to, an optical laboratory management system 200.

The optical laboratory management system 200 is designed to assist in the operations of an optical laboratory that fulfills optical lens orders. For example, the optical laboratory management system 200 may be configured or designed to process prescriptions, keep breakages and errors to a minimum, and provide calculations for the latest lenses.

In accordance with the disclosed embodiment, the optical laboratory management system 200 includes instructions for enabling an end user of the optical laboratory management system 200 such as, but not limited to, a lab technician or lab manager to customize an application user interface of the optical laboratory management system 200. In the depicted example, these instructions for enabling the customization of the application user interface of the optical laboratory management system 200 are encapsulated or grouped into a software module or application referred herein as App Commander 206. In one embodiment, the app commander 206 includes a Header Editor 210, a Palette Editor 220, and a Template Field Editor 230.

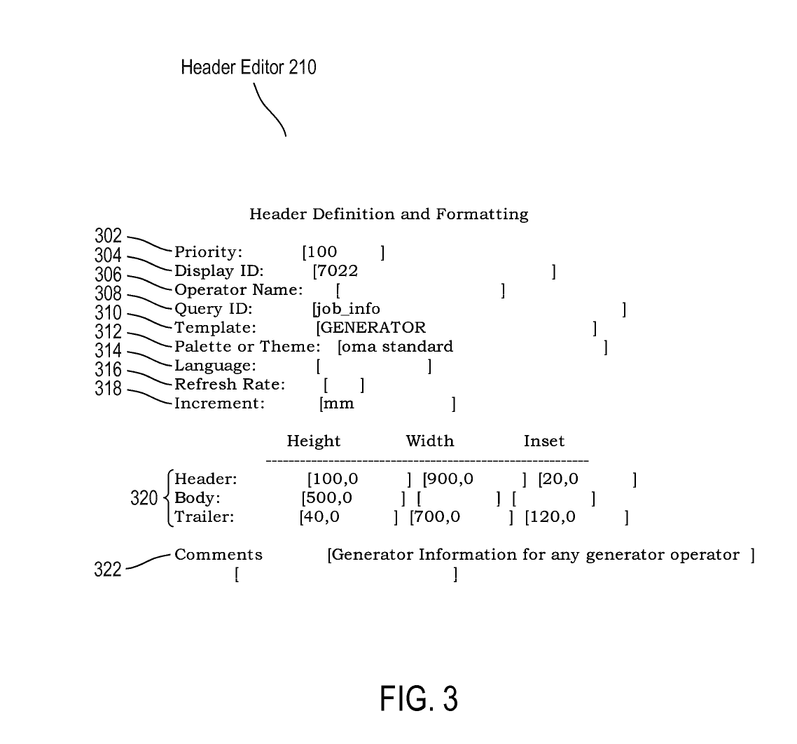

FIG. 3 illustrates an example of a Header Editor 210 in accordance with one embodiment. In one embodiment, the Header Editor 210 is configured to initially define an application and to set up some basic settings for that application. The application is the calling program that provides the requested information on the user interface being generated by the end user. In the depicted embodiment, the Header Editor 210 includes a Priority Field 302, a Display ID Field 304 and an Operator Name Field 306, a Query ID Field 308, a Template ID Field 310, a Palette or Theme Field 312, a Language Field 314, a Refresh Rate Field 316, an Increment Field 318, a Size Field 320, and a Comments Field 322.

In one embodiment, the Priority Field 302 is used by the application to search through a plurality of header entries to find one that fits the qualifications of the calling program and operator. The Display ID Field 304 and Operator Name Field 306 are used to select a heading for the given application. The term heading or header as used herein means heading of a displayed page whether specifically designed as a header or used as a general concept of a page header. The Display ID Field 304 is used to specify a station number or work station at the optical lens processing lab.

In one embodiment, the calling program or start-up menu will acquire a login identity for the user of the application and a choice for the Display ID Field 304 of the application the operator would like to launch. In one embodiment, the App Commander 206 application searches records from a database or data table containing heading data for particular jobs and stops with the first combination that matches both the Display ID Field 304 and Operator Name Field 306. For instance, in one embodiment, during start-up, the App Commander 206 application will query the database in order of the priority of records in the header table of the database. For each record that's read from the database, it will test the data in the Display ID field of the data record against the entry in the Display ID Field 304 that's provided from the calling program or start-up menu. If the two match, then it will compare the login identity of the user that's provided by the calling program or start-up menu to the data in the Operator Name Field 306 of that same data record. If the Operator Name field of the data record matches the login identity provided or if the Operator Name field of the data record is null, the App Commander 206 application will accept this record and launch the application identified by the Display ID Field 304. If the Display ID in the data record does not match the entry in the Display ID Field 304 that's provided by the calling program or start-up men, the application will continue to the next data record in the database. Similarly, if the Display ID's match and the Operator Name Field 306 is non-null and the data in the Operator Name field of the data record do not match the login identity, the App Commander 206 application will continue to the next data record in the database. If all records of the table have been fetched and no matches found, a message to that effect will be returned to the calling program or start-up menu. As an example, the combination of the specified Display ID Field 304 and Operator Name Field 306 may return a record to a template or theme that is specific to a given operator at a specific station.

As stated above, in some embodiments, either the Display ID Field 304 or Operator Name Field 306 may be left blank. For example, in the depicted example illustrated in FIG. 3, a station number is entered for the Display ID Field 304 without specificity to an operator. In this example, the search will return heading records for the specified station number irrespective of an operator. If an operator is specified in the Operator Name Field 306 and the Display ID Field 304 is left blank, then the search will return heading records for the specified operator irrespective of a station number. In some embodiments, the operator may have varying access level to different information. For example, an operator with supervisory permissions may be granted access to additional information that other operators might not be privileged to access such as sales or profit numbers or pricing information.

The Query ID Field 308 allows an administrator to link a specific data set to the application. For example, in the depicted example, the data set “job info” is specified in order to show data on a specific lens order to an operator. The disclosed embodiments may include a plurality of pre-defined data sets that may be specified by the end user and supporting program logic to extract the data when necessary. For example, a user may specify the data set “lens sku info” for an inventory application. Additional non-limiting examples of data sets may include financial data (e.g. sales, cost, profit, credits, outstanding balances), system health (e.g. state of backups, processes running, free drive space), monitoring data (e.g. work-in-process counts, jobs in, jobs shipped, late jobs) and more.

The Template ID Field 310 may be used to identify a name of a template that will be defined by an administrator using the Template Field Editor 230, as will be further explained below. Briefly, the Template Field Editor 230 defines locations and actions of text, data values, prompts, etc. that are to be part of the application that displays the screen to the operator. For example, the screens can be configured to prompt for specific data elements. For instance, to find a specific order, the system may be configured to prompt for the order's job number, tray number, access code, or combination of account and invoice numbers. An administrator can create the template ID name and associate it with the fields using the Template Field Editor 230.

The Palette or Theme Field 312 may be used to specify a palette or theme name that was created by an administrator using the Palette Editor 220. As will be further explained, the Palette Editor 220 can be used to configure or define default colors, font and other parameters that will be used to display the screen to an operator.

The Language Field 314 may be used to specify a language (e.g. French or Spanish) to be used in the display of the screen. This is especially useful when associating a display ID with an operator name so that the operator can see the text titles translated in his or her preferred language. For example, one blocker operator might speak or prefer text in German, while the blocker operator across the aisle from him might only speak or prefer text in Croatian. Each can have his screen translate in real time to the appropriate language.

The Refresh Rate Field 316 may be used to specify how often the screen or displayed information is updated. For instance, this feature is applicable to screens that are intended to be monitor screens for data that will be generated without input from the operator through the App Commander 206 application. For example, these applications are useful for production situations where the operator scans the tray number to a production device such as a blocker or edger. This monitor feature allows the app commander screen to refresh with new data for the scanned tray barcode without requiring a second scan of the barcode into the App Commander 206 application. For example, in production, an operator might scan a barcode of an order at a generator device. That device will submit a request for information from the lab management application. When the lab management application delivers the requested data to the generator device, it can also output a ‘job info’ data set and call the App Commander 206 application to generate a new html output that will be displayed to the screen when the auto-refresh occurs.

The Increment Field 318 is the increment of measure that the browser is to use in drawing the header, body, and trailer boxes with measurements indicated in fields below. In one embodiment, the increment is specified in mm (millimeters) or px (pixels). However, the disclosed embodiments can be adapted to other units of measure that are recognized by the browser(s) or other display mechanism.

The Size Field 320 specifies the height, width and inset measurements that can be added to define location and size of header, body, and trailer boxes on the display in the unit of measure defined by increment above.

In one embodiment, a Comments Field 322 may be used by an administrator to provide reference notes that is used when querying or editing records in the future. Other types of comments or notes may also be added to any record.

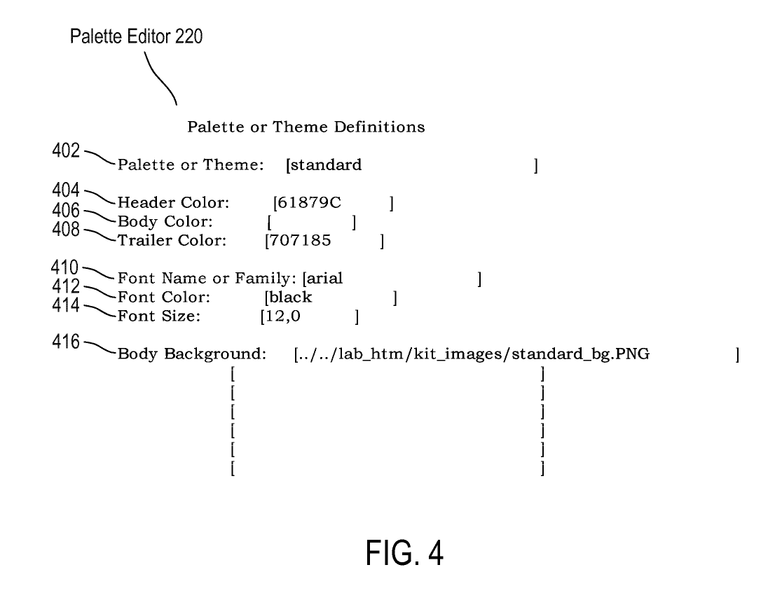

FIG. 4 illustrates an example of a Palette Editor 220 in accordance with one embodiment. The Palette Editor 220 allows an administrator to define one or more themes that can be associated with different Display ID's for different applications.

In one embodiment, the Palette or Theme field 402 can be a name created by the administrator and simply added to the Header Editor 210 on any one or more records that should display the given palette.

The Header Color field 404, Body Color field 406, and Trailer Color field 408 are used to specify the colors to be used by the browser or other display mechanism when displaying the header, body, and trailer boxes as defined in the Header Editor 210. The values such as the 61879C represent the colors recognized by the browser or display mechanism. Though this example shows a 6 character RGB combination any sequence recognized by the display mechanism can be used.

Similarly, the Font Name or Family field 410 and Font Color field 412 are used to define the font design and color to be used by the display mechanism. The font design and color are subject to the browser or display mechanism's ability to represent the given font.

The Font Size field 414 is used to specify the font size in points and may be restricted by the ability of the browser to represent the font size for a given font name or family specified.

The Body Background field 416 specifies the location of an image that the application uses as a background image. For example, in one embodiment, the Body Background field 416 may specify a Uniform Resource Locator (URL) path to a picture or other image that the browser can render as a background. Any type of image format recognized and supported by the browser or display mechanism, such as, but not limited to, Joint Photographic Experts Group (JPEG) or Portable Network Graphics (PNG), may be specified. In one embodiment, if a background URL is specified, the body color field 406 is disregarded or may be left empty because the background image will be displayed in place of any body color in the body of the display.

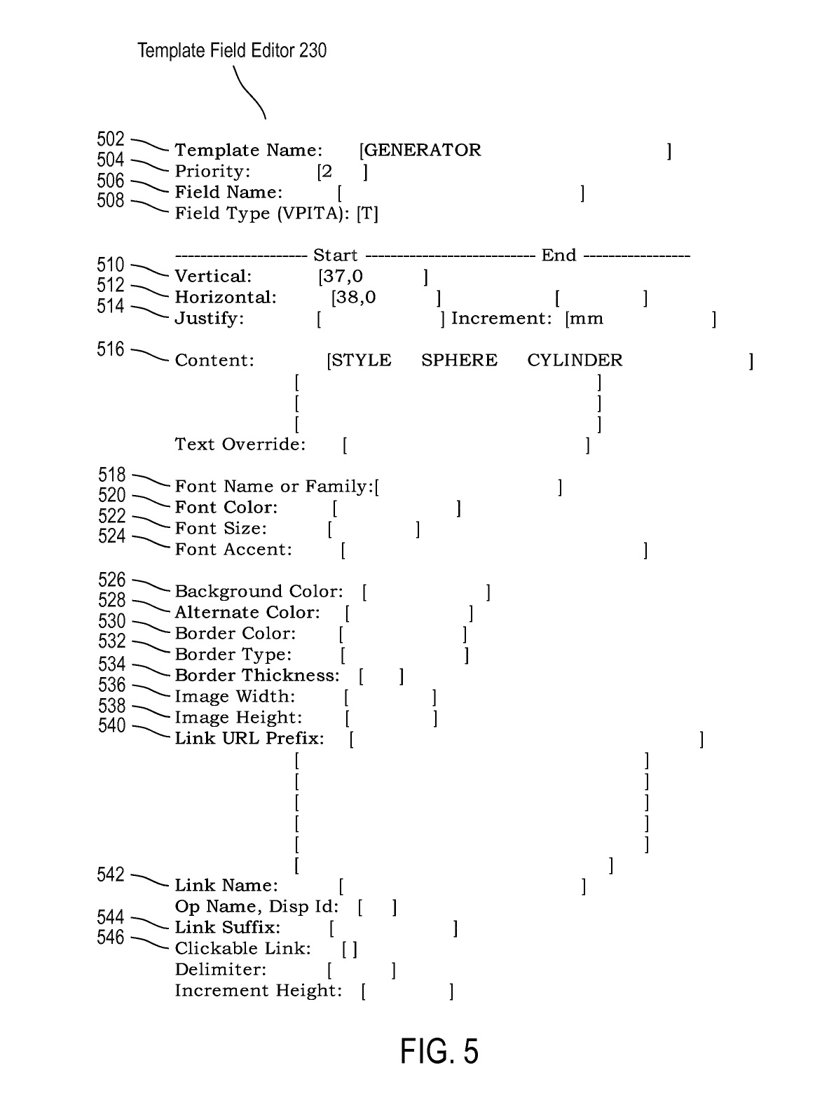

FIG. 5 illustrates an example of a Template Field Editor 230 in accordance with one embodiment. In one embodiment, the Template Field Editor 230 is used to position text, data values, prompts, and images in the display and to format them. Additionally, in some embodiments, the Template Field Editor 230 may be used to effect actions to be executed by the App Commander 206 program. In certain embodiments, formatting for colors or fonts for specific fields may override the default formats for colors and fonts that were previously defined in the palette editor.

The Template Name field 502 and Priority field 504 ties back to the Template ID Field 310 and Priority Field 302 specified in the Header Editor 210. The Field Name field 506 is used to specify a reference name that can be used by the App Commander 206 application to act upon the data element that's associated with the given template record at time of execution. Actions may include but are not limited to summing up data elements with like reference names, or applying multipliers or other actions according to formulas specified as part of the App Commander 206 application and its configuration.

The Field Type field 508 is used to specify the type of action or content that is specified in a Content Field 516. This field type dictates the functions of the application user interface. For example, in the depicted embodiment, the Field Type field 508 indicates a “T” for text. In other embodiments the field type might specify a prompt, image, or data value.

In one embodiment, the Vertical, Horizontal, and Justify fields 510, 512, 514 along with the Increment field 516 indicate the positioning of the text relative to the top left corner of the display. The disclosed embodiments may position the text relative to any point on a display in other embodiments.

In the depicted example, the Content Field 516 specifies that the text words Style, Sphere, and Cylinder be displayed on a screen.

The Font Name or Family field 518, Font Color field 520, and Font Size field 522, and Font Accent field 524 are configured to enable an individual record of the Template Field Editor 230 to override font settings that were assigned in the Palette Editor 220. In one embodiment, the Font Accent field 524 allows entries such as bold, underline, or italic. Any or all of these fields can be applied to any given record.

The Background Color field 526, Alternate Color field 528, Border Color field 530, Border Type field 532, Border Thickness field 534, Image Width field 536, and Image Height field 538 are fields for fields for configuring the various settings of the background color or background image.

The fields for Link URL Prefix field 540, Link Name field 542, Link Suffix field 544 are all concatenated together to form the URL. For example, the Link URL Prefix field 540 contains the text “https://www.”, the Link Name field 542 contains the text “olsssystems” and the Link Suffix field 544 contains the text “.com” and when concatenated together forms the URL: “https://www.olsssystems.com”. The Clickable Link field 546 may be used to indicate to the system that it should configure this for the browser or display mechanism as a clickable link and display the text in the Content Field 516 for the operator to click.

Referring back to the Field Type field 508, in some embodiments, the Field Type field 508 can also be configured with a type “V” to indicate a value. For instance, in one embodiment, when a value (“V”) is configured, the system will use the Content Field 516 to match against the data that have been selected by the system to show to the operator. For example, if a prompt of tray number had allowed the system to find a ‘job info’ data set for a given tray, the selected data set for that tray would be compared to the content value in each record of the data set that's retrieved from the database to see if a match could be found.

In one embodiment, the data set will be represented as key-value pairs with a title or key on the left side of a line and the value to the right. For example, below is a short segment of an example data set for one order in accordance with one embodiment.

RIGHT MIN BLANK DIAM: 69.94

LEFTMINBLANKDIAM:69.94

RIGHTRECOMMENDEDBLOCKSIZE:60.00

LEFTRECOMMENDEDBLOCKSIZE:60.00

RIGHTBLOCKSHOULDERTHICKNESS:11.8

LEFTBLOCKSHOULDERTHICKNESS:11.8

RIGHTPRISMBASEDIRECTION:DWN

LEFTPRISMBASEDIRECTION:DWN

RIGHTBLOCKEDPRISM:1.09

LEFTBLOCKEDPRISM:1.09

RIGHTPRISMAXIS:270

LEFTPRISMAXIS:270

Using the above example dataset, if the Field Type field 508 is specified as type “V” and the Content Field 516 specifies “RIGHTRECOMMENDEDBLOCKSIZE:”, then the App Commander 206 would match the Content Field 516 value of the record above with the key on the left side of the records of the data set. In the above given dataset, the App Commander 206 would find a match as indicated by the bolded key-value pair above and parse the value from the right side of that same line of the data set. It would then display the value 60.0 that it had parsed in the vertical and horizontal positioning specified with right justification within the horizontal boundaries specified. Although the above example dataset uses key-value pair, in other embodiments, other formats for the dataset may be used such as, but not limited to, data sets in xml.

In some embodiments, the Template Field Editor 230 can also be used to configure prompts that the operator can fill in when the application user interface is displayed in production. For example, in one embodiment, a “P” in the field type field 508 indicates to the system that it is to prompt the user based on the Content Field 516. For example, the Content Field 516 may contain the phrase “Please Enter a Tray Number:” for prompting a user to enter a tray number.

In one embodiment, if the field type field 508 is type “P”, then the Link URL Prefix field 540 may be used to configure the action to take place once the tray number has been entered. For example, in one embodiment, the Link URL Prefix field 540 may contain the address or file location of a script that is executed (e.g., “/cgi-bin/nps_dir/prompt_script.sh”). For example, in one embodiment, the configured script will be executed to select data to display based on the entered tray value. The Link Name field 542 is used to represent the entered data to the executed script (e.g., prompt_script.sh) or other specified program, with the expectation that the given script or program can understand the meaning of the data entered for the prompt and act upon it accordingly. For example, in one embodiment, in response to receiving a tray number entry, the script is configured to cause a display to show the order parameters for a new tray. As another example, in response to receiving a sku number entry, the script may be configured to display a bin location and quantity on hand for a given lens with that sku number.

In one embodiment, the prompt value is associated with the Query ID field 308 by the App Commander 206 application and the appropriate data set is selected using the available prompt data to select results from the database. The App Commander 206 will form the data into a data set of key-value pairs that can then be compared to the various template V (Value Field Type) entries and displayed anew with the newly selected and parsed values. In other examples, the received user input or entries can be used to cause the system to update a database. For example, if a prompt exists to capture tray notes, the system can trigger a database update that would insert tray notes that are then associated with the given tray. In one embodiment, these tray notes will be visible to customer service after the update occurs.

Various other actions might be configured within the system using the disclosed embodiments include actions to put an order on hold, cancel an order, other system-defined actions, or user-defined actions configured within the App Commander 206 application. For example, in one embodiment, the App Commander 206 may be built with a set of possible actions that it can effect. In one embodiment, these actions will be constrained to insure that appropriate parameters are provided by the application user interface that the user has built. For instance, a single tray that's currently in production must be specified by the operator if he or she would like to put that tray on hold. If the operator is unable to supply proper qualifications for the chosen action, then, in one embodiment, the system will report an error to the operator.

Still, in certain embodiments, the App Commander 206 may be configured with prompt sequences for beginning and ending account numbers, beginning and ending date range, and a record with an Action or “A” entry for the Field Type field 508 that could generate a report. For example, the system may be configured to generate a statistical report for a certain customer regarding a particular type of lens orders within a certain date range. For reporting, a set of prompts can be configured to capture a range of accounts and dates and to launch a third party reporting system such as Varilux Statistics™, Essilor Digital Report™ or other reporting systems that have been configured to accept prompts from this innovative new process. Other type of actions may include, but not limited to, configuring the system to display an image or play a video such as a training video to a particular operator or at a particular work station.

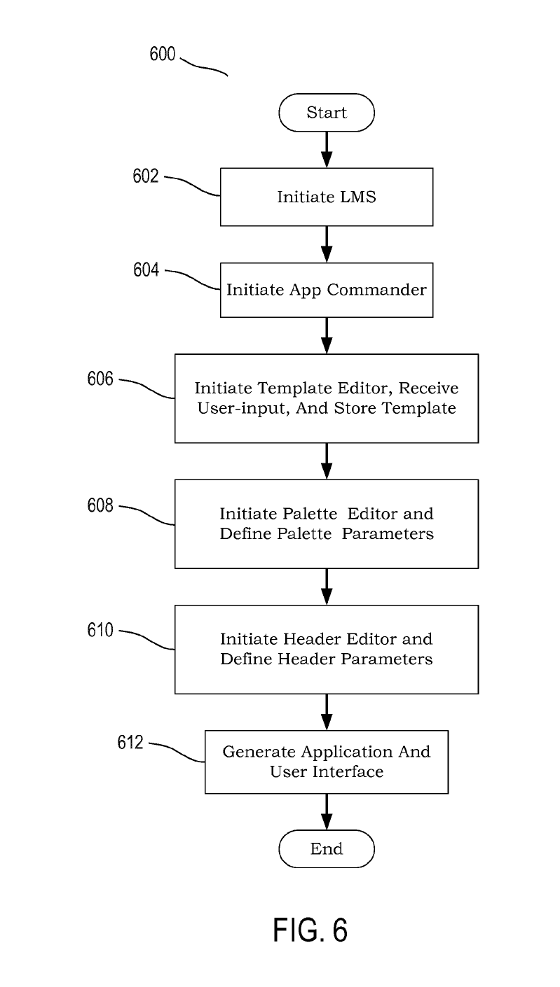

FIG. 6 is a flowchart illustrating a process 600 for customizing a user interface of an optical lab management system in accordance with one embodiment. In accordance with the depicted embodiment, the process begins with initiating the lab management system at step 602. Once the lab management system is up and running, an end-user can initiate the App Commander application at step 602 to create and customize his/her own application and user interface (application user interface) within the lab management system. In one embodiment, the App Commander application can be initiated by selecting an icon or can be initiated by the user issuing a run command in a command window or could be launched directly through a login procedure.

The process presents the user with a Template Editor at step 604. The process receives the user-input into the Template Editor as described above and stores it as a template.

At step 606, the process initiates the Palette Editor. The process receives the user-input into the Palette Editor as described above and stores it as a palette.

At step 608, the process initiates the Header Editor. The process receives the user-input into the Header Editor as described above and stores it as a header. The process then generates the user created application user interface at step 612. The process displays the user created interface on a display at the workstation associated with the user, with the process 600 ending thereafter. This same process may be repeated to create and generate other application user interfaces within the lab management system as desired by a user.

The above process enables an end-user (e.g., a lab operator or administrator) to create and customize his/her desired application user interface without requiring the assistance of the software development team. For example, using the disclosed embodiments, an administrator is able to configure and maintain a variety of screen configurations that suit the business or even liven up a dull interface. For example, in one embodiment, an administrator can devise a set of background colors and images that follow holiday themes such as Easter, Halloween, and Christmas. By simply updating the referenced theme in the header record using the Header Editor for any operator(s), the color scheme and pictures can change with the theme.

The above process is just one example of creating a user-customized application user interface in connection with a lab management system. The above process is not intended to restrict the process of the creating and generating a user interface as disclosed herein. For instance, step 602 only has to be performed if the lab management system is not up and running. Similarly, once the App Commander application is initiated, more than one application user interface may be created without reinitiating the App Commander application each time. Further, the each of the templates, palettes, and header may be created at earlier times and stored by the system for later use. Thus, the process does not require that a template, palette, or a header be created anew each time a user creates an application user interface. Still, in some embodiments, the disclosed embodiments may be a separate, distinct program from the lab management system. For example, the App Commander application may be a third party program that is configured with application program interfaces (APIs) to be able to communicate with one or more different types of lab management systems.

In addition, although three separate editors have been disclosed herein, other embodiments may combined one or more the editors into one, or split one or more of the editors into additional substitute-editors. Further, although the above embodiments depict textual editors for creating and generating user-customized application user interface for a lab management system, other embodiments may use more of a graphical interface for enabling a user to create user-customized application user interface. For example, in one embodiment, a graphical user interface (GUI) may be presented to a user in which the user can drag and drop or graphically (e.g., using a mouse pointer) indicate locations of images or text. In some embodiments, a drop down menu may be presented to the user to enable the user to define actions or other parameters that will be performed by the application.

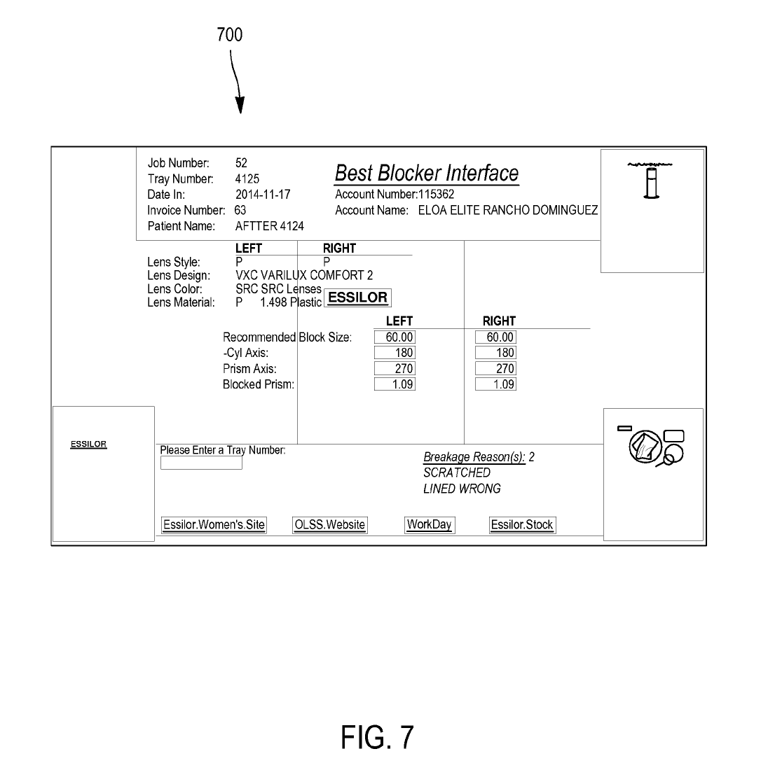

FIG. 7 illustrates an example of a user-created application user interface 700 that is generated in accordance with one embodiment. In the depicted embodiment, application user interface 700 demonstrates a fairly standard theme for a blocker operator with three different pictures configured to show in the corners and a central picture to show a corporate logo behind the working data.

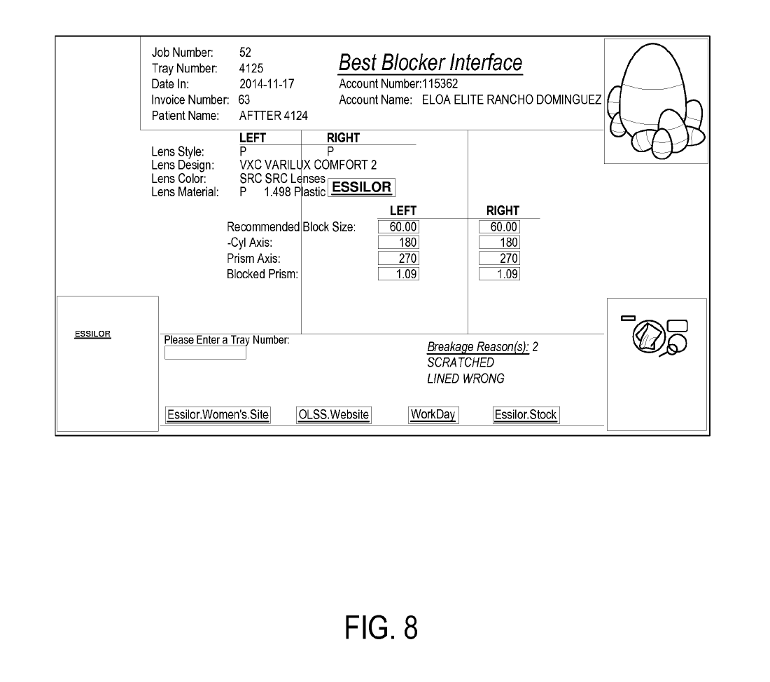

FIG. 8 illustrates an example of a holiday theme applied to the user-created application user interface shown in FIG. 7 in accordance with one embodiment. Again, this may be done by simply applying a different palette or theme using the Header Editor 210 once the palette/theme is created using the Palette Editor 210. The palettes and themes can be managed at the lab level, group level (e.g. a theme for blockers, another for customer service, another for generator operators), individual level, or a mix. Pictures can be represented based on station, operator, group or other level. The pictures can represent corporate initiatives, group initiatives, sports themes or personal preferences (e.g. pictures of the user's children or pets).

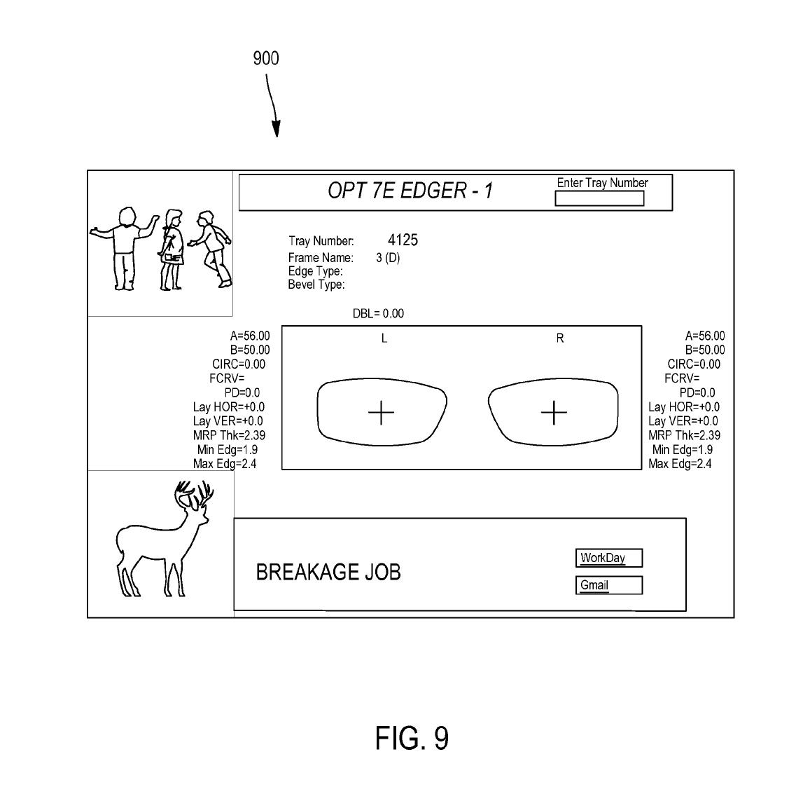

FIG. 9 illustrates another example of a user-created application user interface 900 that is generated in accordance with one embodiment disclosed herein. In the depicted embodiment, the application 900 displays personalized family photos or other images on the user interface along with data associated with a lens order. For example, the application 900 displays the tray number, frame name, edge type, and other lens information associated with the lens order. As shown, in the depicted embodiment, the user can enter a tray number, either manually or by using a barcode reader to scan a tray barcode, to retrieve the information associated with the tray number. In other embodiments, the user may enter in a lens order number or other types of parameters associated with a lens order such as, but not limited to, a date range or certain type of lens for retrieving and displaying information about lens orders.

In the depicted embodiment, the breakage history, if any, associated with the displayed order is shown to the operator. This enables the operator to be aware of extenuating circumstances associated with the order. For example, the breakage history may indicate that the lens is difficult to manufacture and more care should be taken at one or more processing stations. The breakage history may also indicate that the lens order is late, due to past breakages, and that the operator should expedite this order.

Further, as depicted in the application 900, internal or external links to other system or data may be included on the user-created application interface. For example, the system can be configured to provide clickable links to external sites such as a knowledgebase, email, VisionWeb™, or EyeFinity™. Links can also point to internal business sites such as WorkDay™ or Kronos™ for HR activities or other corporate sites. Link may also point to instructional videos or documents that are related to the lens order, operator, or work station. In one embodiment, these clickable links can be configured to be conditional based upon data found in the selected data set being represented on the screen. For example, a station configured for a finishing blocker might represent a link titled “Slippery Coat” that is displayed any time it displays an order that includes one of several anti-reflective coatings that present special difficulties for the finishing department. The link might point to a document that explains clearly the best practice for managing orders with these coatings in the finishing department.

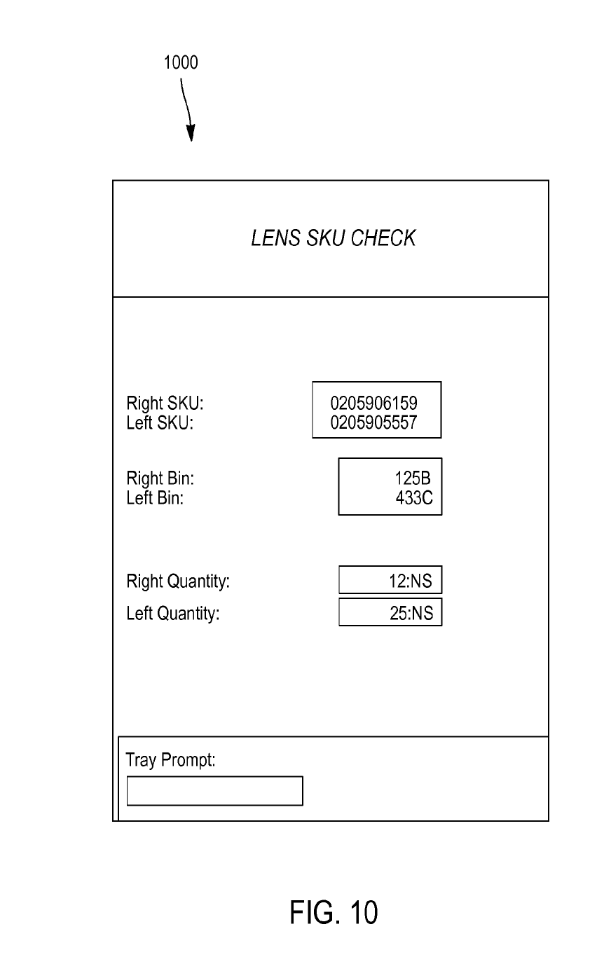

FIG. 10 illustrates another example of user-created application user interface 1000 that is generated in accordance with one embodiment disclosed herein. The user-created application user interface 1000 is configured to display lens order data based on the size of a display of a mobile device such as, but not limited to, a smart phone. Using the disclosed embodiments, the screens can be configured to meet the sizes of a mixture of display devices (e.g., such as a large over-head monitor, a desktop PC, a laptop, a tablet, or a cell phone) and altered by an administrator as needed when an operator might purchase a new device without need for intervention by a software developer or program release. In fact, all of the above displays and many more can be configured with these three editor screens and require no additional development by a software developer. The administrators can configure the screen design, prompts, images, text headers and values without need of a program update or change. The screens can be altered during production without need to stop production or commit to new program implementations. It supports themes, corporate pictures and links along with personal pictures and internal and external links to one or more systems or websites.

Accordingly, the disclosed systems and methods of the present application provide lab users and lab administrators an effective tool to create a wide variety of useful screen and applications that fit the needs of the individual business, group or user without the typical software development cycle. In accordance with one embodiment, the App Commander allows users or administrators of the laboratory or commercial entity to design their own applications (apps) with a series of editors that will not require intervention by a software developer. For instance, the disclosed embodiments provide a series of database editors that can be used to define the look and actions of a data entry, look-up, or monitor screen. As used herein the term “database editor” means an editor, interface, or program that is configured to enable a user to edit or retrieve the content of a database. The database editor is not limited to the look or design disclosed herein. The database editors allow the administrator the capability to define the use or purpose of the screen, the specific data fields and titles represented on the screen, and the set of data to use in selecting the specific fields to be able to tune the applications and screens to fit specific needs of the lab, stations, or users within the lab. The editors allow configuration of prompts that can be used to both qualify the data set selected for display and prompts that can be used to effect programs that perform specific actions or database updates. This innovation allows real-time access to a wide variety of data elements, and data sets, and abilities to configure the screens to act upon those data elements to offer links, warnings and suggestions based on the selected data. For example, special services on the order can be configured to appear only when present and to appear in a variety of ways including large fonts, bright font or back-ground colors and links to support documents or videos that can help a new or even experienced operator perform a new or challenging service.

In addition, the screens can be configured to display data for specific orders including equipment settings, warnings, and services; groups of orders including history, status, tray notes, and services; inventory data such as bin locations, quantities on hand, quantities on order, and cost; and financial data for specific accounts for the business or for specific sales territories, system health including back-up status and last successful back-up date/time, indicators for background processes that have stopped working, and warnings when system is low on drive space. For financial data, prompts for beginning and ending company, branch, territory, or account could be set up along with beginning and ending date ranges. For inventory, the system can be configured to prompt for and receive a sku number and/or bin number. In addition, in some embodiments, if a specific order has been identified by entering one or more of the prompts above, the screen could be configured to allow the operator to choose to put the tray “on hold” or to cancel the order or to prompt for and then update tray notes with a note keyed by the operator.

Further, the editors have sufficient controls to allow users or administrators to design and effect use of the application screens without the need for intervention by a software developer. For instance, the screen design, themes, color palettes including pictures and back-grounds can all be managed by the system administrator or even end user. The disclosed embodiments are very versatile and will meet needs for a wide range of uses without requiring that a user or lab administrator have an understanding of HTML or other programming languages to be effective at creating useful and appealing applications for a range of devices.

Additionally, in some embodiments, the screen designs and ideas can be shared or collaborated upon using a collaboration portal or other sharing means to offer ideas and starting points for users and administrators. In this way, the design process becomes more social and other users/lab administrators can provide their input.

The above disclosed embodiments have been presented for purposes of illustration and to enable one of ordinary skill in the art to practice the disclosed embodiments, but is not intended to be exhaustive or limited to the forms disclosed. Many insubstantial modifications and variations will be apparent to those of ordinary skill in the art without departing from the scope and spirit of the disclosure. For instance, although the flowcharts depict a serial process, some of the steps/blocks may be performed in parallel or out of sequence, or combined into a single step/block. The scope of the claims is intended to broadly cover the disclosed embodiments and any such modification.

As used herein, the singular forms “a”, “an” and “the” are intended to include the plural forms as well, unless the context clearly indicates otherwise. It will be further understood that the terms “include”, “including”, “comprise”, and/or “comprising,” when used in this specification and/or the claims, specify the presence of stated features, steps, operations, elements, and/or components, but do not preclude the presence or addition of one or more other features, steps, operations, elements, components, and/or groups thereof. In addition, the steps and components described in the above embodiments and Figures are merely illustrative and do not imply that any particular step or component is a requirement of a claimed embodiment.

Additionally, although specific terms are employed herein, they are used in a generic and descriptive sense only and not for purposes of limitation. For instance, the term database, as used herein, is intended to include any form of organized data, including, but not limited to, data found in tables, charts, spreadsheets, and documents. Furthermore, the term database does not imply the use of a particular or specialized database software nor does it imply the use of any particular data structure.

What is claimed is:

1. An optical lab management system comprising:
memory configured to store computer executable instructions and data;
a network interface configured to enable the optical lab management system to communicate data with at least one networked device; and
a processor for executing the computer executable instructions, wherein the computer executable instructions comprises instructions for:
providing at least one database editor configured to enable an end user to design an application user interface of the optical lab management system, wherein the at least one database editor includes at least one configuration field for defining a layout of the application user interface and for defining specific data fields to be displayed on the application user interface.

2. The optical lab management system of claim 1, wherein the at least one database editor includes at least one configuration field for defining a set of data to use in selecting the specific data fields.

3. The optical lab management system of claim 1, wherein the at least one database editor includes at least one configuration field for defining at least one of a use and purpose of the application user interface.

4. The optical lab management system of claim 3, wherein the at least one database editor includes at least one configuration field for defining titles represented on a screen.

5. The optical lab management system of claim 1, wherein the at least one database editor includes at least one configuration field for including clickable links to at least one of external sites and internal sites on the application user interface.

6. The optical lab management system of claim 5, wherein the clickable links are configurable to be conditional based upon data found in a selected data set being represented on the application user interface.

7. The optical lab management system of claim 1, wherein the at least one database editor includes at least one configuration field for defining a screen design, theme, color palette, and background image of the application user interface.

8. The optical lab management system of claim 7, further comprising configuring the screen design for representation on one of a wide range of devices including a large over-head monitor, a desktop PC, a laptop, a tablet, and a cell phone.

9. The optical lab management system of claim 7, further comprising selectively applying the theme can be to one of a lab level, a group level, and at an individual level.

10. The optical lab management system of claim 1, wherein the at least one database editor includes a palette editor for configuring a look of the application user interface, a template field editor for configuring a functionality of the application user interface, and a header editor for defining the application user interface using a created palette and theme.

11. A computer-implemented method for customizing an application user interface of an optical lab management system, the method comprising:
configuring a look and function of an application user interface of an optical lab management system using at least one database editor configured to enable an end user to design the application user interface of the optical lab management system.

12. The computer-implemented method of claim 11, wherein configuring the look and function of the application user interface of the optical lab management system comprises defining a screen design, theme, color palette, and background image of the application user interface.

13. The computer-implemented method of claim 12, wherein configuring the look and function of the application user interface of the optical lab management system comprises defining the screen design based on a size of a display of a mobile device.

14. The computer-implemented method of claim 12, wherein configuring the look and function of the application user interface of the optical lab management system comprises configuring clickable links to at least one of external sites and internal sites on the application user interface, wherein the clickable links are configurable to be conditional based upon data found in a selected data set being represented on the application user interface.

15. A non-transitory computer-readable storage media having stored thereon computer-executable instructions, that when executed by a processor of a system, performs:
configuring a look and function of an application user interface of an optical lab management system using at least one database editor that is configured to enable an end user to design the application user interface of the optical lab management system, wherein configuring the look and function of the application user interface of the optical lab management system comprises defining a screen design, theme, color palette, background image, and defining specific data fields to be displayed on the application user interface.
